(19) United States
(12) Patent Application Publication
(10) Pub. No.: US 2005/0090372 A1
(43) Pub. Date: Apr. 28, 2005

(54) Method and system for using a database containing rehabilitation plans indexed across multiple dimensions

(76) Inventors: Mark Burrows, Bryn Mawr, PA (US); John Cronin, Milton, VT (US); Tushar Narsana, Winooski, VT (US); John Anthony Singarayar, Skillman, NJ (US)

Correspondence Address: NORRIS MCLAUGHLIN & MARCUS, P.A., P O BOX 1018, SOMERVILLE, NJ, 08876, US

(21) Appl. No.: 10/876,172

(22) Filed: Jun. 24, 2004

Related U.S. Application Data
(60) Provisional application No. 60/482,876, filed on Jun. 26, 2003.
(60) Provisional application No. 60/482,159, filed on Jun. 24, 2003.

(51) Int. Cl. A63B 71/00; A61B 5/00

(52) U.S. Cl. 482/8

(57) ABSTRACT

A searchable database contains rehabilitation plans for individuals having the same medical condition. The rehabilitation plans are indexed by scores corresponding to physical dimensions and personal environment dimensions characteristics of the respective individuals having the same medical condition and also by scores representative of actual success results associated with prescribing the respective rehabilitation plans. The database can be queried to identify rehabilitation plans for a dimension whose assessment information matches individual assessment information for the same dimension of an individual requiring rehabilitation for the same medical condition. In addition, the actual success scores can be used to identify rehabilitation plans in the database likely to be successful when prescribed to an individual. The individual assessment information and the identified rehabilitation plan prescribed to the individual, and also feedback obtained from the individual to which an identified plan is prescribed and also success results for the prescribed plan, can be used to update the database.

### CROSS REFERENCE TO RELATED APPLICATIONS

This application claims the benefit of U.S. Provisional Application Nos. 60/482,876 filed Jun. 26, 2003 and 60/482,159 filed Jun. 24, 2003, assigned to the assignee of this application and incorporated by reference herein. The subject matter of U.S. patent application Ser. No. ______, filed Jun. 24, 2004 and entitled “METHOD AND SYSTEM FOR REHABILITATING A MEDICAL CONDITION ACROSS MULTIPLE DIMENSIONS”, assigned to the assignee of this application, is related to this application.

### FIELD OF THE INVENTION

The present invention relates generally to rehabilitation of an individual having a medical condition and, more particularly, to using a database containing historical information from results of rehabilitation plans previously prescribed to patients having a same medical condition to select a rehabilitation plan for an individual also having the same medical condition, where the database includes rehabilitation plans indexed by physical dimensions and personal environment dimensions characteristics of patients to which the rehabilitation plans were respectively prescribed and by actual success results.

### BACKGROUND OF THE INVENTION

It is well known that rehabilitation of many medical conditions, such as hearing loss, central nervous system conditions, high cholesterol, diabetes, obesity, etc., usually requires an individual with a medical condition to perform certain actions that are designed to alleviate the condition, but will not necessarily result in curing or eliminating the condition. For example, an individual suffering from the medical condition of hearing loss is unlikely to ever have perfect hearing again, despite the use of an instrument, such as a programmable hearing aid, that may be prescribed to address the physical aspects of the condition. The approach that the medical community currently takes to rehabilitate a medical condition∝such as hearing loss, is to prescribe an instrument that addresses only some of the physical aspects of the medical condition. The instrument is designed to address the physical aspects of the medical condition, so as to alleviate the medical condition sufficiently to enable the individual to attain what most persons would consider to be a more normal lifestyle. The instrument will successfully address the physical aspects of the medical condition so long as the individual uses in the instrument in the manner specifically required by the instrument.

It has been recognized that the unique and personal characteristics of an individual can significantly impact whether an individual will adhere to a rehabilitation plan that includes the use of an instrument designed to treat physical aspects of the condition. The unique and personal characteristics of an individual, or an individual's personal environment dimensions, can include, for example, the individual's preferences and dislikes, tendencies, psychological profile and the like. By formally integrating the results of an assessment of an individual's unique and personal characteristics and also the individual's physical characteristics into the selection of a rehabilitation plan for a medical condition, an individual is more likely to adhere to a prescribed rehabilitation plan and, therefore, succeed in rehabilitating the medical condition. In such circumstances, the prescribed treatments and the rehabilitation goals are tailored to the individual's unique and personal characteristics and also physical characteristics to obtain a customized, overall rehabilitation program. For a customized rehabilitation program, the individual is more likely to use an instrument that addresses physical aspects of the condition in the required manner, while also performing other treatments that are part of the rehabilitative program.

U.S. patent application for “METHOD AND SYSTEM FOR REHABILITATING A MEDICAL CONDITION ACROSS MULTIPLE DIMENSIONS”, Ser. No. ______, filed Jun. 24, 2004 (“MULTIPLE DIMENSIONS”), assigned to the assignee of this application and incorporated by reference herein, describes method and system for accounting for the unique and personal characteristics of an individual, or the so-called personal environment dimensions, and the physical characteristics of the individual, or the so-called physical dimensions, when selecting a rehabilitation plan that involves the use of an instrument to address physical aspects of the medical condition so as to develop a customized rehabilitation program that is likely to successfully attain the rehabilitation goals of the individual. In summary, the “MULTIPLE DIMENSIONS” application defines diagnostic metrics based on measurement information obtained from assessment of respective physical dimensions and personal environment dimensions of an individual having a medical condition and, using the metrics, generates from the measurement information diagnostic and goal scores that can be used to select a rehabilitation plan for the individual. The selection of a rehabilitation plan using the diagnostic and goal scores corresponding to an individual's respective assessed dimensions characteristics can include searching a database which includes, for each of a plurality of physical dimensions and personal environment dimensions that can impact rehabilitation of the medical condition, previously prescribed rehabilitation plans indexed by respective diagnostic scores, where the scores represent the results of assessments of the physical dimensions and personal environment dimensions of patients previously prescribed the corresponding rehabilitation plans.

It is known that historical information concerning an individual's responses to, and the actual results of, implementation of any medical treatment program, such as a rehabilitation plan selected by use of scores representative of assessed dimensions characteristics of an individual as described in the “MULTIPLE DIMENSIONS” application, will be generated over time. A collection of this information can provide a health care professional with an opportunity to leverage valuable insights and knowledge gained from patient responses to prescribed treatments so that an improved a treatment program can be developed. For example, in clinical trials for pharmaceutical drugs, a pharmaceutical company performs continual and iterative testing of patients, stores test results in a database, and modifies and improves new tests based on prior test data stored in the database to ultimately arrive at a drug formula that is ready to be mass marketed.

It would be beneficial to a health care professional who prescribes rehabilitation plans from a rehabilitation plan database including rehabilitation plans indexed by respective physical dimensions and personal environment dimensions characteristics, see the “MULTIPLE DIMENSIONS” application, to have a simple way to research actual success results of previously prescribed rehabilitation programs to increase effectiveness of new rehabilitation programs that are prescribed to patients. For example, audiologists would benefit from having the aural rehabilitation database continuously updated with knowledge, such as actual success results, that is gained from prescribing an individual an aural rehabilitation plan selected from the database.

Therefore, there exists a need for a rehabilitation plan database, which contains rehabilitation plans indexed by physical and personal environment dimensions characteristics such as described in the “MULTIPLE DIMENSIONS” application, that can be easily accessed for researching previously prescribed rehabilitation plans and for developing new, more effective rehabilitation plans based on knowledge, such as actual success results, gained from prescribing a rehabilitation plan selected from the database.

### SUMMARY OF THE INVENTION

In accordance with the present invention, a database contains, for each of a plurality of physical dimensions and personal environment dimensions associated with a medical condition, profile information and scores indexing respective rehabilitation plans. The database can be queried from a remote location to identify rehabilitation plans in the database whose respective scores correspond to at least a first of the dimensions and fall within a matching range of a score generated based on the results of an assessment of the same at least first dimension of an individual having the medical condition. The rehabilitation plans stored in the database include rehabilitation plans previously prescribed to patients, and optionally rehabilitation plans generated from performing an interpolation using information from previously prescribed rehabilitation.

The database is updated based on feedback obtained from an individual who is prescribed a rehabilitation plan selected from the matching rehabilitation plans, or to include annotation information representative of progress results concerning the prescribed plan. The updating preferably can include annotating, or adding a new rehabilitation plan record including the prescribed rehabilitation plan indexed by the individual's profile information and score used in the query that resulted in identification of the prescribed rehabilitation plan. In a preferred embodiment, the updating includes indexing a prescribed rehabilitation plan in the database corresponding to a dimension with an actual success score that represents actual treatment success results for the individual to whom the plan was prescribed.

In a preferred embodiment, an aural rehabilitation plan database includes aural rehabilitation plans indexed by scores corresponding to the results of assessments of physical dimensions of hearing loss, such as volume and pitch, and of personal environment dimensions of hearing loss that can impact use of a hearing aid device to address the physical dimensions. The personal environment dimensions can include, for example, speech intelligibility, real-world needs, critical success factors, localization, appearance and performance trade-offs and quality preferences. When the database includes at least a threshold number of stored aural rehabilitation plan records, the database is queried to retrieve a plurality of aural rehabilitation plans having scores that are within a search range and correspond to respective assessed physical dimensions and personal environment dimensions of an individual having hearing loss. The search range is a function of the individual scores generated based on results of the assessment of the dimensions of the individual. From the retrieved aural rehabilitation plans, an aural rehabilitation plan is selected using the scores of the individual for the assessed respective dimensions, and also in view of the individual's profile information, which preferably was obtained during the assessment of the dimensions of the individual. Any annotation information from the database concerning the selected plan is also available for selecting a plan to be prescribed to the individual.

In a preferred embodiment, the annotation information includes an actual treatment success result that indexes the prescribed rehabilitation plan for the corresponding dimension, and the query of the database includes actual success results for a rehabilitation plan corresponding to at least a first dimension, in combination with a range of scores derived from the assessment of the at least first dimension of the individual, as search criteria used to retrieve rehabilitation plans from the database.

In a further preferred embodiment, the query of the database includes individual profile information and a cumulative individual diagnostic score, in addition to a range of scores derived from the assessment of at least a first dimension of the individual, as search criteria used to retrieve rehabilitation plans from the database. The cumulative individual diagnostic score is preferably a sum of all of the individual's diagnostic scores. The retrieved plans are identified in rank order, based on cumulative actual success scores for the individual prescribed the retrieved plan corresponding to the first dimension, where the cumulative actual success score is preferably a sum of all of the actual success scores for the individual to whom the retrieved plan was prescribed. In addition, the ranking in order is also based on the actual success score for the retrieved rehabilitation plan corresponding to the first dimension.

### BRIEF DESCRIPTION OF THE DRAWINGS

Other objects and advantages of the present invention will be apparent from the following detailed description of the presently preferred embodiments, which description should be considered in conjunction with the accompanying drawings in which like references indicate similar elements and in which:

FIG. 1 is a system diagram of an audiologist prescribing an aural rehabilitation plan to an individual with hearing loss using an aural rehabilitation plan database of a central computer hearing health system in accordance with the present invention.

FIG. 2 is a representative database table for use in developing an aural rehabilitation program for an individual in accordance with the present invention.

FIG. 3 illustrates a preferred method of prescribing an aural rehabilitation program to an individual with hearing loss using the computer system of FIG. 1.

FIGS. 4A, 4B, 4C, 4D and 4E are exemplary representative database tables that the computer system of FIG. 1 identifies in response to a query.

### DETAILED DESCRIPTION OF THE INVENTION

The present invention provides for system and method for using a rehabilitation plan database, which contains rehabilitation plans indexed by respective physical dimensions and personal environment dimensions characteristics, such as described in the “MULTIPLE DIMENSIONS” application, to select a rehabilitation plan for an individual having a medical condition, in view of the individual's dimensions characteristics and also actual success results associated with the respective rehabilitation plans of the database. For purposes of highlighting the features of the present invention, the present inventive technique is described below in connection with the selection of an aural rehabilitation plan for an individual having hearing loss from an aural rehabilitation plan database, where that database contains previously prescribed aural rehabilitation plans indexed by respective physical dimensions and personal environment dimensions characteristics of the patients to which the aural rehabilitation plans were prescribed and actual treatment success results for the prescribed plans, and where the selected aural rehabilitation plan preferably includes the use of a programmable hearing aid. The database optionally includes aural rehabilitation plans generated from performing an interpolation using information from previously prescribed aural rehabilitation plans. It is to be understood that the present inventive technique can be readily applied to selecting a rehabilitation plan for any of various medical conditions, such as, for example, central nervous systems conditions including Alzheimer's disease, cardiological conditions, high cholesterol, joint replacement, diabetes, hypertension, schizophrenia and other like medical conditions, for which an instrument likely will be prescribed to address physical aspects of the condition to alleviate the condition at least in part.

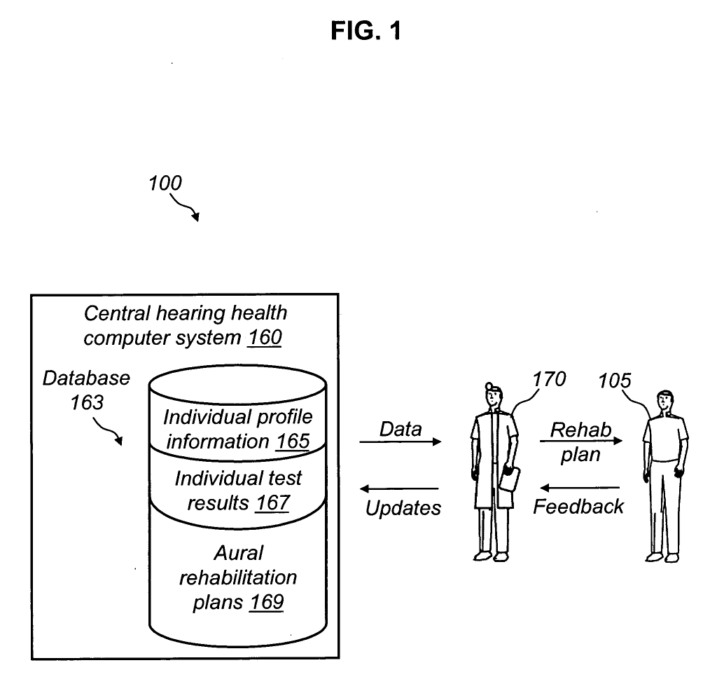

FIG. 1 illustrates a preferred embodiment of a system 100 for prescribing an aural rehabilitation plan to an individual 105 in accordance with the present invention. Referring to FIG. 1, the system 100 includes a central hearing health computer system 160 and an audiologist 170 who is treating the individual 105. The central hearing health computer system 160 includes a database 163. The database 163 contains a plurality of individual profile information 165, a plurality of individual test results 167 and a plurality of aural rehabilitation plans 169. Audiologist 170 is a highly trained and licensed hearing health professional, such as a physician, and is preferably certified to prescribe a remedy, such as an aural rehabilitation plan, for the individual 105.

The central hearing health computer system 160 is a centrally located computer system that is capable of performing all normal computer functions, such as reading and writing data to database 163, reading and writing data to a display monitor (not shown), communicating through networks and executing programs to access and use data stored in database 163.

Database 163 is a central database repository within central hearing health computer system 160. The data stored within database 163 can be classified into three main areas: individual profile information 165, individual test results 167 and aural rehabilitation plans 169. Individual profile information 165 is profile information including, for example, an individual's name, contact information, age and career profile. Individual test results 167 are the individual's hearing test results, such as diagnostic scores and goal scores for the individual 105 generated from measurement information obtained from assessment of physical and personal environment dimensions of the individual 105. Aural rehabilitation plans 169 stores the aural rehabilitation plans that are prescribed to patients, such as the individual 105, in accordance with the present invention as described in further detail below.

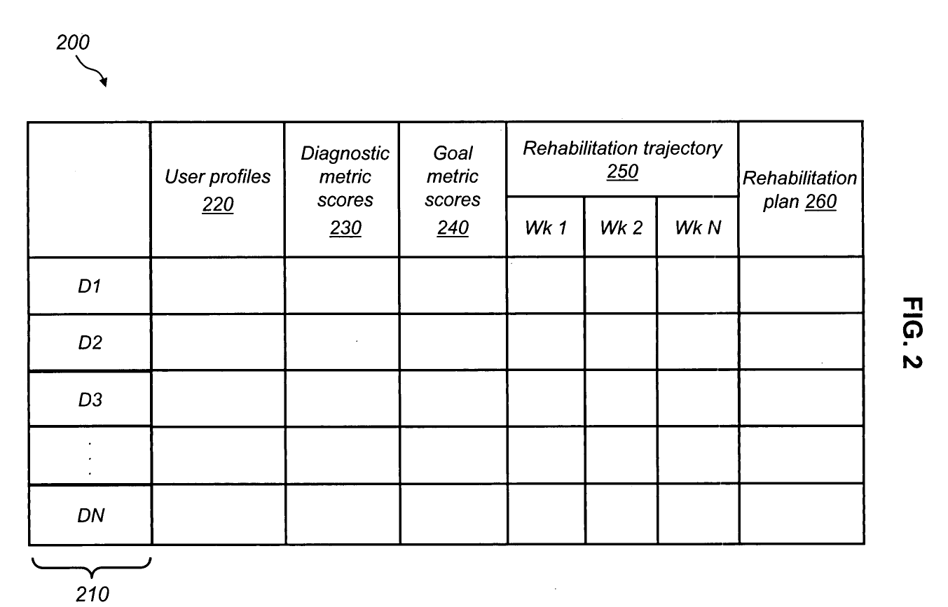

The plans 169 of the database 163 further include aural rehabilitation plan records. Each record identifies a previously prescribed aural rehabilitation plan based on profile information and test results for the patient to whom the plan was prescribed and also the dimension of the patient to which the test results correspond, and further identifies the rehabilitation trajectory associated with the plan. In addition, in a preferred embodiment, a record includes annotation information representing actual treatment success results for the individual which are obtained, such as by re-assessing the individual, following completion of a prescribed plan by the individual. Optionally, a record identifying an interpolated aural rehabilitation plan can be generated by performing an interpolation using information from previously prescribed rehabilitation plans. The records are evaluated, in accordance with the present Inventlon as described in further detail in the text accompanying the description of FIGS. 2, 3 and 4A-4F, to select an aural rehabilitation plan for an individual with hearing loss. For ease of reference and clarity in explaining the present invention, the records in the plans 269 are described below as containing previously prescribed rehabilitation plans.

In accordance with the present invention, the audiologist 170 selects a previously prescribed rehabilitation plan for the individual 105 by searching the aural rehabilitation records in the database 263. The selecting involves comparing the individual profile information and also the test results corresponding to at least a first of the assessed physical dimensions and personal environment dimensions for the individual 105 with the previously prescribed rehabilitation plans of the records in the plans 269 indexed by the same first assessed dimension, and then selecting, from the compared aural rehabilitation plans, an aural rehabilitation plan having matching profile information and scores. The selecting further can include using the actual success results information for the individuals to whom the respective retrieved plans were previously prescribed to rank a plurality of selected plans.

FIG. 2 illustrates an exemplary virtual database table 200 that preferably is contained within the aural rehabilitation plans 169 of the database 163 and constitutes compilations of aural rehabilitation plan records containing previously prescribed rehabilitation plans. There are preferably a plurality of tables 200 for respective patients in the database 263. Referring to FIG. 2, each table 200 includes aural rehabilitation plans 260 indexed by diagnostic metrics 210 and also corresponding user profile information 220, diagnostic metric scores 230 and goal metric scores 240. The table 200 further includes the rehabilitation trajectory 250 for each of the rehabilitation plans 260.

Diagnostic metrics 210 correspond to assessments of physical and personal environment dimensions of an individual with hearing loss. Typical diagnostic metrics 210 corresponding to assessed dimensions of an individual having hearing loss include a volume metric, a pitch metric, a speech intelligibility metric, a real-world needs metric, an individual's critical success factors, a localization metric, an appearance and performance trade-off metric and a quality preference metric. See “MULTIPLE DIMENSIONS” application for a detailed description of the dimensions that correspond to these metrics.

User profiles 220 contain the profile of the patient to whom the rehabilitation plan 260 was prescribed for a corresponding diagnostic metric 210. Diagnostic metric scores 230 are the scores computed in view of the diagnostic metrics 210, where the metrics 210 are defined based on measurement information obtained from assessments of respective dimensions of patients prescribed respective rehabilitation plans 260. Goal metric scores 240 are the best scores the patient can expect to achieve with assisted hearing, given the extent of hearing loss. In a preferred embodiment, the audiologist assigns a goal metric score 240 for a metric 210 based on, for example, the profile of the patient and the diagnostic score 230 for the metric. In an alternative preferred embodiment, for a diagnostic metric 210, the goal metric score 240 is the same as the diagnostic score 230.

Rehabilitation trajectory 250 is the step-by-step progress that the patient should make to proceed from diagnostic metric score 230 to goal metric scores 240 for each diagnostic metric 210. Rehabilitation trajectory 250 further includes WK1, WK2, through WKN, which represent the expected progressive improvement of the patient with hearing loss on a weekly basis from the first week to the Nth week, where N varies and is the number of total weeks for rehabilitation prescribed to the patient.

Rehabilitation plan 260 is the rehabilitation plan previously prescribed to a patient with hearing loss to restore hearing loss in one or both ears and to progress from diagnostic metric scores 230 to goal metric scores 240 along rehabilitation trajectory 250.

In a preferred embodiment, the database 163 is a relational database having the ability to store hearing loss data, such as data contained in the table 200, with minimal duplication and to link or relate data from different sources, thereby providing that the table 200 can be formed through known database programming techniques such as structured queries.

In a further preferred embodiment, the plan 260 for a dimension 210D can include a score representative of actual treatment success of the rehabilitation plan for the individual to whom the plan was prescribed. The actual success score is used to index the rehabilitation plan within the database. For example, the actual success score can constitute a diagnostic score generated based on assessment of the individual after the individual completes the treatment set forth by the prescribed rehabilitation plan corresponding to the dimension 210D.

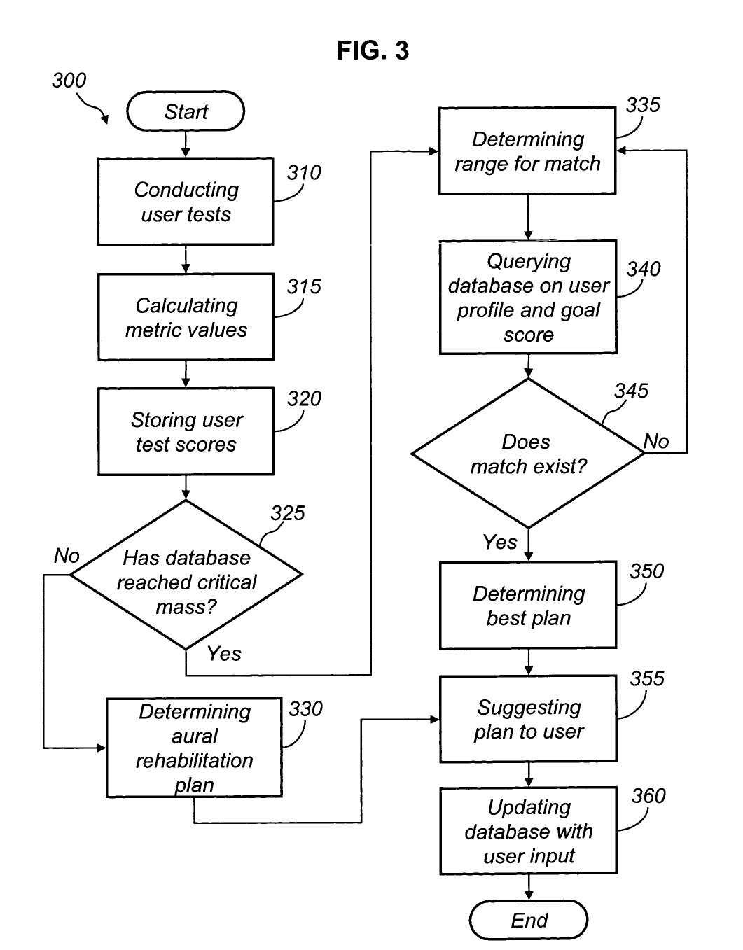

In accordance with the present invention, audiologist 170, following a high level method similar to the exemplary preferred method 300 set forth in FIG. 3, supplies individual profile information and individual test results for an individual 105 to the computer system 160. Based on the profile information and the test results, the system 160 evaluates the previously prescribed aural rehabilitation plans, which are represented in records forming the database tables 200 of the database 163, to identify suitable aural rehabilitation plans for the individual 105. The audiologist 170 then selects a rehabilitation plan from the identified plans and prescribes the selected plan to the individual 105. In a further preferred embodiment, the audiologist 170 prescribes the selected plan in view of any annotation information included with the selected rehabilitation plan information supplied by the system 160. The individual 105 then provides feedback on the prescribed plan to audiologist 170, who in turn uses the feedback to update aural rehabilitation plan records in the plans 169 in the database 163. The updating can include annotating the record containing the selected rehabilitation plan in the rehabilitation plans 169 to include progress results, an actual success score or the individual's experience with the rehabilitative treatment. Further, the updating can include adding, such as to the tables 200, a new aural rehabilitation plan record that is stored in the rehabilitation plans 169. The new record includes a new aural rehabilitation plan indexed by a diagnostic metric 210; the individual's test results for the metric 210 as reflected in the scores 230 and 240; the individual's profile information for the metric 210 as reflected in the user profiles 220; and, if available, actual success scores that are contained in the plan 260. The new record also reflects the rehabilitation trajectory performed in the rehabilitation trajectory 250.

For purposes of illustration, the method 300 is described in detail below in connection with the use of the system 100 and the table 200. Referring to FIG. 3, in step 310, an audiologist 170 assesses physical and personal environment dimensions of the individual 105 to obtain measurement information needed to define the diagnostic metrics 210 and to generate diagnostic scores and goal scores for the respective metrics 210. For example, an assessment of the personal environment dimension of speech intelligibility can be performed in accordance with a diagnostic testing method of creating a training product customized for an individual that determines specific, troublesome words and sentences based on the individual's hearing profile, and stores the measurement information obtained from such testing in a database. See “SYSTEM FOR AND METHOD OF TRAINING A USER TO UNDERSTAND HUMAN SPEECH CORRECTLY WITH A HEARING AID DEVICE”, U.S. Provisional Application Ser. No. 60/482,159, filed Jun. 24, 2003 and assigned to the assignee of this application and incorporated by reference herein. Further in step 315, the audiologist 170 collects basic profile information for the individual 105 and then stores this information in individual profile information 165.

Following step 310, in step 315 the audiologist 170 defines metrics 210 for the respective corresponding assessed dimensions based on measurement information obtained from assessment of the dimensions. For example, a diagnostic metrics 210 is defined by aggregating individual test measurements for the corresponding dimension to derive a standard. See, “MULTIPLE DIMENSIONS” application, incorporated by reference herein, which describes system and method for quantifying raw measurement information obtained during an assessment of the dimensions of an individual with hearing loss that are associated with volume, pitch, speech intelligibility, real-world needs, individual's critical success factors, localization, appearance/performance trade-offs and quality preferences. The definition of the diagnostic metrics is preferably performed by normalizing the measurement information obtained for thee respective dimensions into a signal metric along a linear scale. Following the definition of a diagnostic metric, a diagnostic score 230 can be generated with reference to the diagnostic metric, for example to the volume metric, by a simple addition of all scores where a perfect hearing score is 100% and worst-case hearing is 0%. In a preferred embodiment, an audiologist can assign different weights to each diagnostic metric 210. In addition, in step 315, the audiologist 170 may use the measurement information corresponding to a metric 210 to define a goal metric score 240.

Following step 315, in step 320 the audiologist 170 causes diagnostic metric scores 230 and any goal score 240 for the individual 105 for each respective diagnostic metric 210 to be stored in the individual test results 167 of the database 163. In addition, the system 260 uses the metric specific diagnostic and goal scores for the individual 105 to generate a virtual table 200 for the individual 105. A counter (not shown) is incremented at the end of step 315, which signifies the end of entry of testing information belonging to the current individual 105 in a table 200. Following step 355, as discussed in detail below, the prescribed aural rehabilitation plan corresponding to a diagnostic metric and the rehabilitation trajectory for such plan are inserted into the aural rehabilitation plan records in the table 200 for the individual 105.

Following step 320, in step 325, the audiologist 170 determines whether there is a critical mass of occurrences for completed tables 200 within database 163, i.e., whether the counter in step 320 has reached a greater than satisfactory level. For example, audiologist 170 can determine that critical mass exists when the counter indicates that there are more than 10,000 occurrences of table 200 in the database 163. If critical mass has not been reached, the method 300 proceeds to step 330.

Step 330 is performed because the audiologist 170 considers the database 163 does not have significant historical information concerning previously prescribed aural rehabilitation plans. In step 330, the audiologist 170, therefore, develops an aural rehabilitation plan for the individual 105 based on the audiologist's own experience and knowledge, such as clinical practice experience and textbook research. The process 300 then proceeds to step 355.

Alternatively, if critical mass has been reached in step 325, the method 300 proceeds to step 335 following step 325. In step 335, audiologist 170 determines a range of scores, such as a range of diagnostic scores, a range of goal scores or both the former and latter, for searching the tables 200 in database 163 so that a broadly gathered, highly focused dataset of aural rehabilitation plans that are suitable for the individual 105, in view of the individual's test results and profile information, is identified. For example, if the individual's diagnostic score for the speech intelligibility metric is at 45% of its potential value, audiologist 170 can construct a search query to provide that the database 163 is searched only for previously prescribed aural rehabilitation plans that have a diagnostic score for the speech intelligibility metric as low as 40% and as high as 50%.

Following step 335, in step 340 audiologist 170 supplies a query to the system 160 that causes the system 160 to evaluate the tables 200 in the database 163 to retrieve previously prescribed rehabilitation plans constituting matches for the specific individual 105. The query causes the system 160 to compare individual profile information 165 and individual test results 167 for the individual 105 with user profiles 220, a range of diagnostic metric scores 230 and a range of goal metric scores 240 in the tables 200 of the database 163. The system 160 is likely to retrieve a plurality of previously prescribed rehabilitation plans from the database 163, because the search query includes a range of scores to be searched for respective corresponding diagnostic metrics 210. The plurality of previously prescribed rehabilitation plans retrieved from the search permits the audiologist to determine a more effective aural rehabilitation plan for the individual 105.

Continuing with the exemplary search range described in connection with step 335, the system 160 in step 340, based on the search query requirement that only plans having diagnostic scores for the speech intelligibility metric in the range of 40% to 50% be identified, retrieves a plurality of rehabilitation plans from the tables 200 of the database 163 matching the search criteria. The system 160 supplies the audiologist 170 with the matching occurrences of multiple records of previously prescribed rehabilitation plans in a format similar to table 200. The system 160 can utilize any well known prior art technique one of matching user profiles and scores from tables within a database, such as, for example, U.S. Pat. No. 6,063,028, incorporated by reference herein.

In a preferred embodiment, a software application such as a simple Visual Basic form can be used as a GUI screen for audiologist 170, who enters the desired parameters for querying database 163. The application then converts the parameters into a standard database query that the system 160 can use to search the tables 200 of the database 163. In an alternative embodiment, if the audiologist 170 is technologically savvy, the audiologist can bypass the application and simply write a query against database 163 by using, for example, the Structured Query Language (SQL) to retrieve selected previously prescribed aural rehabilitation plan information. Although query languages and methods of writing queries are highly dependent on the format of a database 163, any well known prior art technique can be used in accordance with the present invention depending on the database 163 format.

Following step 340, in step 345 the audiologist 170 determines whether the query specified in step 340 has returned a satisfactory match for the individual 105. For example, if at least five matching aural rehabilitation plan records are retrieved, then audiologist 170 is satisfied that sufficient historical data is available to develop an aural rehabilitation plan for the individual 105. If a match exists, the method 300 proceeds to step 350. If not, method 300 returns to step 335, and the audiologist 170 modifies the search range. Steps 335 and 340 can be iterated as many times as needed to obtain a match in step 345.

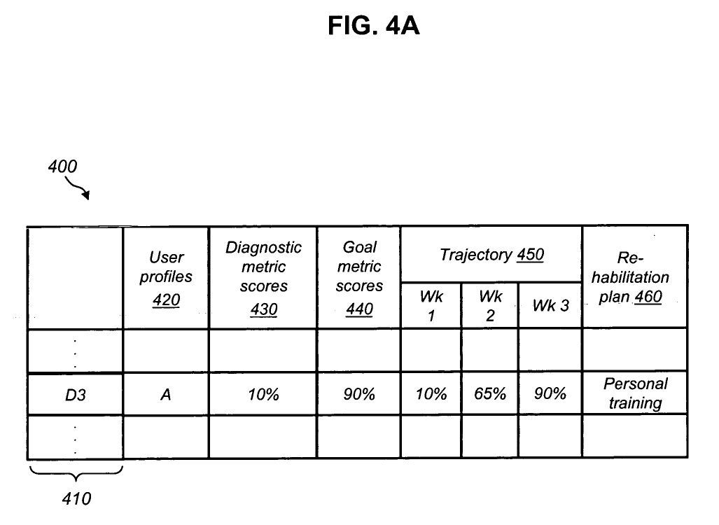

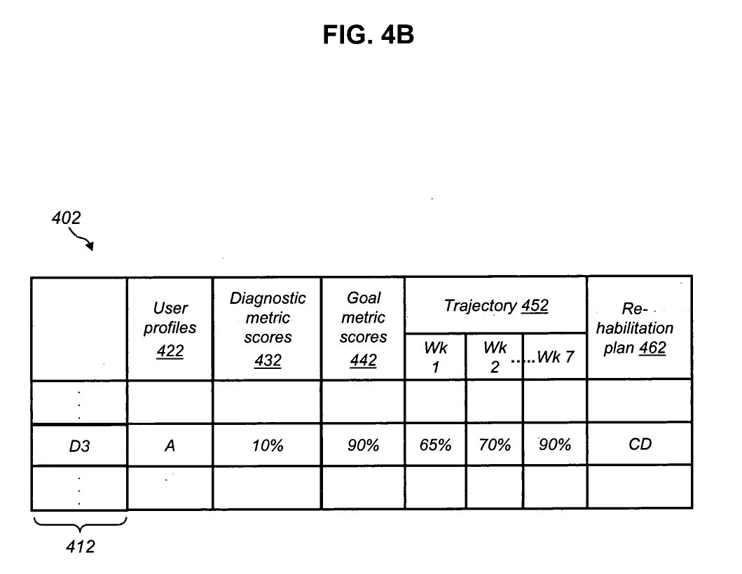

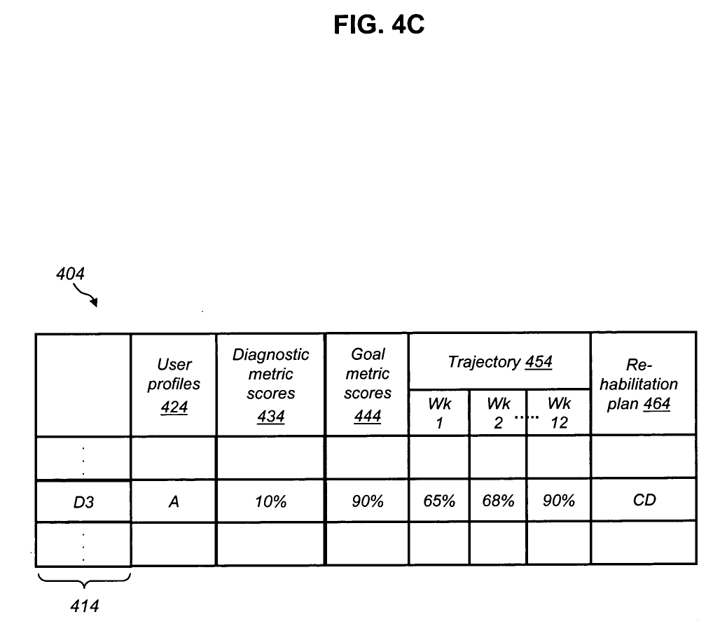

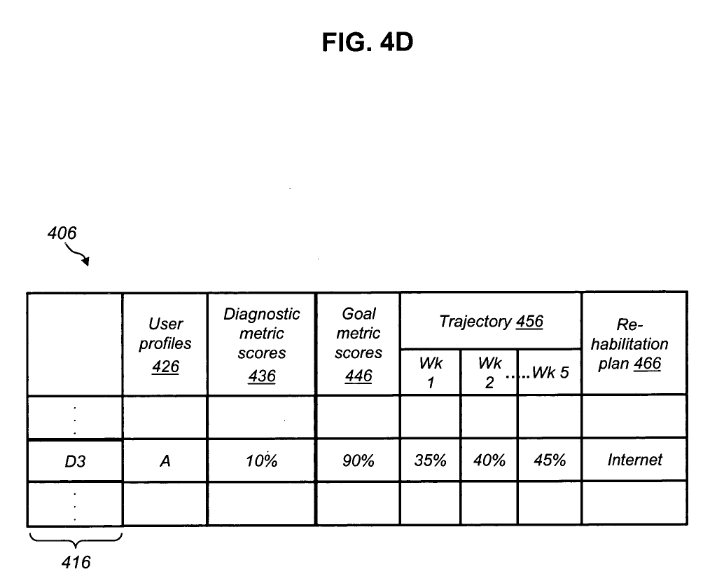

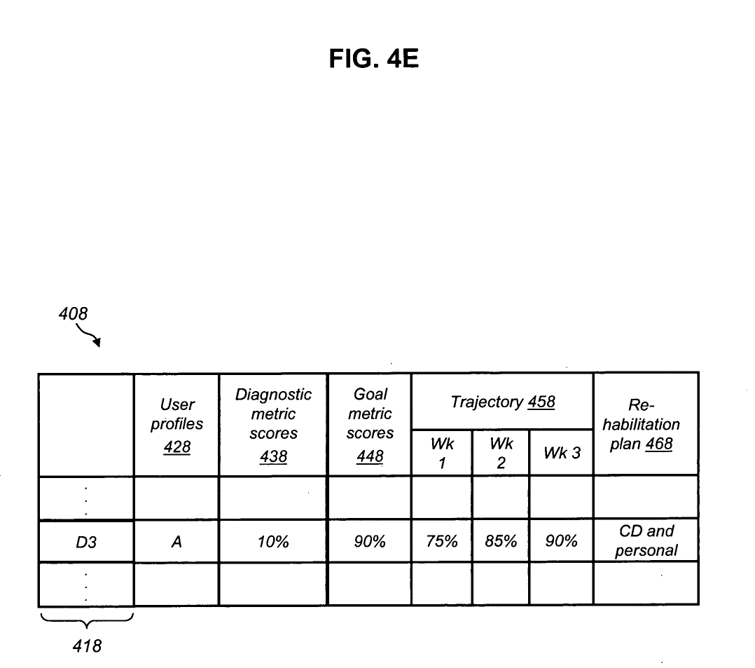

If a match exists in step 345, the audiologist 170 in step 350 determines the best plan for the individual 105. For example, the audiologist 170 may desire to determine the shortest rehabilitation plan for the individual 105 to improve D3 diagnostic metric 210. Based on the search query used in step 340 to retrieve records from the database 163, the system 160 has identified from the aural rehabilitation plans 169 five matching previously prescribed aural rehabilitation plans that may be suitable for the individual 105 and retrieves these records as part of respective tables 400, 402, 404, 406 and 408, as shown in FIGS. 4A-4E. For ease of reference, each of the tables 400, 402, 404, 406 and 408 has the same format as the table 200 discussed above.

Referring to FIGS. 4A-4E table 400 includes a set of diagnostic metrics 410, a plurality of user profiles 420, a plurality of diagnostic metric scores 430, a plurality of goal metric scores 440, a rehabilitation trajectory 450 and a rehabilitation plan 460. Table 402 includes a set of diagnostic metrics 412, a plurality of user profiles 422, a plurality of diagnostic metric scores 432, a plurality of goal metric scores 442, a rehabilitation trajectory 452 and a rehabilitation plan 462. Table 404 includes a set of diagnostic metrics 414, a plurality of user profiles 424, a plurality of diagnostic metric scores 434, a plurality of goal metric scores 444, a rehabilitation trajectory 454 and a rehabilitation plan 464. Table 406 includes a set of diagnostic metrics 416, a plurality of user profiles 426, a plurality of diagnostic metric scores 436, a plurality of goal metric scores 446, a rehabilitation trajectory 456 and a rehabilitation plan 466. Table 408 includes a set of diagnostic metrics 418, a plurality of user profiles 428, a plurality of diagnostic metric scores 438, a plurality of goal metric scores 448, a rehabilitation trajectory 458 and a rehabilitation plan 468.

Continuing with the example, user profiles 420, 422, 424, 426, and 428 are the same values as the user profiles 220 included in the table 200 for the individual 105, and goal metric scores 440, 442, 444, 446, and 448 are the same values as goal metric scores 240 included in the table 200 for the individual 105.

After audiologist 170 analyzes the tables 400, 402, 404, 406 and 408, the audiologist determines that table 408 has diagnostic metric score 438 that is very close to diagnostic metric score 230 of the individual, and that trajectory 458 indicates the shortest rehabilitation time. Therefore, audiologist 170 decides to suggest rehabilitation plan 468 for the individual 105.

Following step 350, audiologist 170 in step 355 prescribes the rehabilitation plan 468 chosen in step 350. Alternatively, in step 355 the audiologist prescribes the rehabilitation plan determined in step 330. As part of step 350, the audiologist 170 identifies to the system 160 the plan selected from step 350, or provides information representative of the plan determined in step 330, such that the records in the table 200 for the individual 105 can be populated with rehabilitation trajectory 250 and rehabilitation plan 260 data for the corresponding diagnostic metric 210. The table 200 for the individual 105, thus, includes a new rehabilitation plan record in the rehabilitation plans 169 in the database 163 that will be considered in a search subsequently entered by another audiologist. In addition, the table 200 for the individual 150 also specifically can be retrieved by the audiologist 170 for further research and study for assessing an overall rehabilitation program, which includes a plurality of selected aural rehabilitation plans, for the individual 105.

In a preferred embodiment, audiologist 170 prescribes to the individual 105 rehabilitation plan 468, now identifiable as rehabilitation plan 260 in the table 200 for the individual 105, over several counseling sessions. Each counseling session marks a milestone in the improvement of the hearing of the individual 105, such that at the end of the final counseling session, the hearing of the individual 105 is at goal metric score 240 for the corresponding diagnostic metric 210. Moreover, to ensure patient satisfaction, in all of the counseling sessions, the individual 105 can provide feedback concerning the treatment method constituting the rehabilitation plan 260, and also the rehabilitation trajectory 250, based on the individual's experience.

For example, if audiologist 170 determines that rehabilitation plan 468 corresponding with diagnostic metric 210 D3, which in the example corresponds to speech intelligibility, is suitable for the individual 105, then audiologist 170 prescribes the treatment represented by the plan 468. The plan 468 can constitute personal hearing training combined with use of a pre-recorded compact disc that teaches the individual 105 to listen and train his brain to interpret an improved version of a series of words and sentences to bring the speech intelligibility metric from diagnostic metric score 230 to goal metric score 240. Trajectory 250 may be spread over three weeks, during which audiologist 170 meets with the individual 105 once a week to study learning progression, as well as to receive feedback from the individual 105 on adding or removing training words from the compact disc.

It is noted that the present invention of prescribing a rehabilitation plan selected from plans identified from querying a database, as described in above in connection with the use of the system 100 to perform the method 300, is applicable to an individual having a medical condition regardless if the individual continually uses a prescribed instrument that addresses physical aspects of the medical condition during assessment or rehabilitation.

Following step 355, in step 360 audiologist 170 provides that the database 163 is updated based on feedback received from the individual 105 or progress results noted by the audiologist. The updating of the database 163 can be performed through standard PC input/output devices, such as a keyboard (not shown) and a monitor (not shown), that can be communicatively coupled to the system 160 over a communications network. For example, based on the feedback, the audiologist 170 supplies information to the system 160 that causes the system 160 to annotate the table 200 for the individual 105 with comments as to whether the prescribed aural rehabilitation plan 260 worked well or did not work well. Alternatively, the audiologist 170 supplies information to the system 160 to provide that the system 160 generates a new rehabilitation plan record in the plans 169. The new plan record, for example, includes a modified version of the rehabilitation plan previously prescribed to the individual that reflects the individual's suggestion or adaptations performed during rehabilitation and is indexed by the individual's profile information and test results (scores) for the corresponding diagnostic metric.

In a further embodiment, the feedback can result in the audiologist 170 re-assessing the individual following completion of a prescribed plan, and generating a new record in the table 200 using the newly obtained individual test results and a new rehabilitation plan selected based on the newly obtained individual test results. Thus, the feedback is useful in ensuring the completeness and accuracy of aural rehabilitation plans 169 within database 163.

In an alternative preferred embodiment, conventional and well known interpolation techniques can be used to generate a new aural rehabilitation plan record in a table 200, where the record includes a plan that never has been prescribed to a patient and has a trajectory plan and diagnostic and goal scores for a specific metric determined from aural rehabilitation plan records in the database 163 containing previously prescribed aural rehabilitation plans.

In a further preferred embodiment, the aural rehabilitation plan record in the database 163 corresponding to the rehabilitation plan prescribed to the individual, and including the individual's diagnostic and goal score for the dimension corresponding to the plan, is updated to include an actual success score. The actual success score preferably indexes the rehabilitation plan in the database 163 for searching purposes.

In still another preferred embodiment, actual success scores are included in the database 163 for indexing the respective prescribed rehabilitation plans. In this preferred embodiment, the search steps performed in steps 340, 345 and 350 of the method 300 can be replaced by a searching step where the query includes, for a subject dimension, a range of diagnostic scores and also actual success as searching criteria. The system 160 first searches the database 163 for rehabilitation plans whose diagnostic scores for the subject dimension are within the range set forth in the query to retrieve a first set of rehabilitation plans. The system 160 then, for each of the plans in the first set, compares actual success scores with diagnostic scores to identify favorable success results, and ranks the retrieved plans of the first set from most favorable to least favorable. A favorable success result is, for example, where the actual success score is not less than, equal to or only insignificantly higher than the diagnostic score for the corresponding plan. For example, a retrieved plan from the first set having a diagnostic score of 20 and an actual success score of 80 would be retrieved, and the difference between the two scores is used to rank the plan against other retrieved plans of the first set determined to have favorable success results.

In a further preferred embodiment where actual success scores are included in the database 163 for indexing respective prescribed rehabilitation plans, the search steps performed in steps 340, 345 and 350 of the method 300 can be replaced as follows. The search query used to perform a search of the database 163 includes individual profile information and an individual diagnostic score for at least a first dimension, and also a cumulative individual diagnostic score, as the search criteria. The cumulative individual diagnostic score is the sum of the individual diagnostic scores generated based on measurement information obtained from assessment of the individual for a set of dimensions that include the same dimensions as those used to index prescribed rehabilitation plans in the database 163. Based on the search query, the system 160 retrieves from the database 163 a first set of prescribed plans. The first set contains all prescribed rehabilitation plans in the database 163 that have profile information matching the individual profile information, such as a matching age or range of ages. For each of the plans in the first retrieved set, the system 160 computes a cumulative diagnostic score, which preferably is the sum of all of the diagnostic scores contained in the database 163 for the patient to whom the plan of the first set was prescribed. The system 160 compares the cumulative diagnostic scores associated with the plans in the first set with a cumulative individual diagnostic score for the individual, and retrieves a second set of plans having a cumulative diagnostic score matching or within a range of the cumulative individual diagnostic score. The cumulative individual diagnostic score preferably is the sum of all of the diagnostic scores for the individual associated with the set of dimensions set forth in the database 163. Then, the system 160 computes cumulative actual success scores for each of the plans in the second set. The cumulative actual success score for a plan in the database is preferably the sum of the actual success scores corresponding to the respective set of dimensions for the patient to whom the plan was previously prescribed. The system 160 then compares the cumulative actual success scores with the cumulative diagnostic scores for each of the respective plans of the second set to identify favorable success results, and ranks the retrieved plans of the second set from most favorable to least favorable. A favorable success result is, for example, where the cumulative actual success score is not less than, equal to or only insignificantly better than the cumulative diagnostic score.

Then, the system 160, for the at least first dimension identified in the search query, searches the plans of the second set to identify plans whose diagnostic score for the first dimension matches, or is within a predetermined range of, the individual diagnostic score for the first dimension and retrieves these plans as a third set of plans. For each of the plans in the third set, the system 160 compares the actual success score with the diagnostic score to identify favorable success results, preferably as described in the above preferred embodiment, and ranks the retrieved plans of the third set from most favorable to least favorable. The system 160 then supplies these plans in rank order as retrieved plans for selection by the audiologist. In a further preferred embodiment, the ranking of the plans from the second set is maintained as the dominant ranking in relation to the selection and ranking of the plans from the third set.

Although preferred embodiments of the present invention have been described and illustrated, it will be apparent to those skilled in the art that various modifications may be made without departing from the principles of the invention.

1. A method for selecting a rehabilitation plan for an individual having a medical condition using a rehabilitation plan database, wherein the database includes, for respective physical dimensions and personal environment dimensions associated with the medical condition, profile information and scores corresponding to respective rehabilitation plans, the method comprising:
querying the database to identify a plurality of rehabilitation plans corresponding to at least a first of the physical dimensions and personal environment dimensions and having profile information and a score matching individual profile information and an individual score, wherein the individual profile information and the individual score are representative of measurement information obtained from an assessment of the at least first dimension of an individual having the medical condition, wherein the scores in the database are generated in view of diagnostic metrics defined from measurement information obtained from assessment of respective physical dimensions and personal environment dimensions, wherein the querying includes selecting a range of scores as constituting a match for the individual score; and
supplying rehabilitation plan information corresponding the identified rehabilitation plans.

2. The method of claim 1 further comprising:
updating the database based on feedback generated from prescribing one of the identified plans to the individual with the medical condition, wherein the updating includes at least one of annotating the plan prescribed to the individual, annotating and adding to the database, for the first assessed dimension, a new rehabilitation plan indexed by the profile information and the score of the individual.

3. The method of claim 1, wherein the database includes actual success scores and diagnostic scores indexing the respective plans corresponding to a set of the dimensions included in the database and wherein individual diagnostic scores are generated from measurement information obtained from assessment of the individual for the same set of dimensions included in the database, and
wherein the selecting includes retrieving rehabilitation plans having profile information and a cumulative diagnostic score matching the individual profile information and a cumulative individual diagnostic score, wherein the cumulative diagnostic score is a function of the diagnostic scores for the individual to whom a rehabilitation plan from the database identified as having matching profile information was prescribed, wherein the cumulative individual diagnostic score is a function of the individual diagnostic scores, wherein the retrieved rehabilitation plans are ranked based on cumulative actual success scores for the individuals to whom the retrieved rehabilitation plans were respectively prescribed, and wherein the cumulative actual success score for a retrieved plan is a function of the actual success scores for the individual prescribed the plan.

4. The method of claim 3 further comprising:
further ranking the retrieved plans based on the actual success score for the retrieved plan corresponding to the first dimension.

5. The method of claim 1, wherein the rehabilitation plans in the database are indexed respectively by actual success scores, and wherein the selecting includes ranking the identified rehabilitation plans in view of the difference between the actual success score and the diagnostic score corresponding to the identified rehabilitation plan.

6. A system for selecting a rehabilitation plan for an individual having a medical condition, the system including a controller having communications capabilities and a rehabilitation plan database,
wherein the database includes, for respective physical dimensions and personal environment dimensions associated with the medical condition, profile information and scores corresponding to respective rehabilitation plans, and
wherein the controller, in response, to a search request for identification of a rehabilitation plan, the request including individual profile information and an individual score corresponding to at least a first of the physical dimensions and the personal environment dimensions, searches the database to identify a plurality of rehabilitation plans corresponding to the at least first dimension and having profile information and a score matching the individual profile information and the individual score corresponding to the at least first dimension, wherein the individual profile information and the individual score are representative of measurement information obtained from an assessment of the least first dimension of the individual having the medical condition, wherein the scores are generated in view of diagnostic metrics defined from measurement information obtained from assessment of physical and personal environment dimensions and wherein the searching includes identifying rehabilitation plans for the first dimension having scores within a predetermined range of the individual score; and
wherein the controller supplies aural rehabilitation plan information corresponding the identified rehabilitation plans.

7. The system of claim 6, wherein the controller, based on feedback generated from prescribing one of the identified plans to the individual with the medical condition, updates the database to include at least one of annotations to the plan prescribed to the individual and, for the first dimension, a new rehabilitation plan Indexed by the profile information and the score of the individual.

8. The system of claim 6, wherein the database includes actual success scores and diagnostic scores indexing the respective plans corresponding to a set of the dimensions included in the database and wherein individual diagnostic scores are generated from measurement information obtained from assessment of the individual for the same set of dimensions included in the database, and
wherein the controller searches the database to retrieve rehabilitation plans having profile information and a cumulative diagnostic score matching the individual profile information and a cumulative individual diagnostic score, wherein the cumulative diagnostic score is a function of the diagnostic scores for the individual to whom a rehabilitation plan from the database identified as having matching profile information was prescribed, wherein the cumulative individual diagnostic score is a function of the individual diagnostic scores, wherein the retrieved rehabilitation plans are ranked based on cumulative actual success scores for the individuals to whom the retrieved rehabilitation plans were respectively prescribed, and wherein the cumulative actual success score for a retrieved plan is a function of the actual success scores for the individual prescribed the plan.

9. The system of claim 7 further comprising:
wherein the controller ranks the retrieved plans based on the actual success score for the retrieved plan corresponding to the first dimension.

10. The system of claim 6, wherein the rehabilitation plans in the database are indexed respectively by actual success scores, and wherein the controller ranks the identified rehabilitation plans in view of the difference between the actual success score and the diagnostic score corresponding to the identified rehabilitation plan.

11. A method for selecting an aural rehabilitation plan for an individual having hearing loss using an aural rehabilitation plan database, wherein the database includes, for respective physical and personal environment dimensions associated with hearing loss, profile information and scores corresponding to respective aural rehabilitation plans, the method comprising:
querying the database to identify a plurality of aural rehabilitation plans corresponding to at least a first of the physical and personal environment dimensions and having profile information and a score matching profile information and a score for an individual having hear loss, wherein the individual profile information and the individual score are representative of measurement information obtained from an assessment of the least first dimension of the individual, wherein the querying includes selecting a range of scores as constituting a match for the individual score; and
supplying aural rehabilitation plan information corresponding to the identified aural rehabilitation plans.

12. The method of claim 11 further comprising:
storing a new rehabilitation plan for the at least first dimension at the database, wherein the new rehabilitation plan constitutes one of the identified rehabilitation plans and is indexed by profile information and a score respectively corresponding to the individual profile and the individual score of the individual having hearing loss.

13. The method of claim 11 further comprising:
performing the querying step using updated individual profile and individual score for the at least first dimension, wherein the updated individual profile and individual score are based on an assessment of the physical and personal environment dimensions of the individual performed following prescription of the one identified rehabilitation plan to the individual.

14. The method of claim 11 further comprising:
updating the database using feedback generated based on prescribing one of the identified plans to the individual, wherein the updating includes at least one of annotating the identified plan and adding, for the first dimension, a new aural rehabilitation plan indexed by the profile information and the score of the individual

15. The method of claim 11, wherein the diagnostic metrics are defined by normalizing the measurement information obtained for each of the assessed dimensions into a linear scale signal metric and wherein the generated scores and the scores in the database each comprise at least one of a diagnostic score and a goal score.

16. The method of claim 11, wherein the personal environment dimensions include at least two of speech intelligibility, real world needs, critical success factors, localization, appearance and performance trade-offs and quality preferences.

17. The method of claim 11, wherein the database includes actual success scores and diagnostic scores indexing the respective aural rehabilitation plans corresponding to a set of the dimensions included in the database and wherein individual diagnostic scores are generated from measurement information obtained from assessment of the individual for the same set of dimensions included in the database, and
wherein the selecting includes retrieving aural rehabilitation plans having profile information and a cumulative diagnostic score matching the individual profile information and a cumulative individual diagnostic score, wherein the cumulative diagnostic score is a function of the diagnostic scores for the individual to whom a rehabilitation plan from the database identified as having matching profile information was prescribed, wherein the cumulative individual diagnostic score is a function of the individual diagnostic scores, wherein the retrieved aural rehabilitation plans are ranked based on cumulative actual success scores for the individuals to whom the retrieved rehabilitation plans were respectively prescribed, and wherein the cumulative actual success score for a retrieved aural rehabilitation plan is a function of the actual success scores for the individual prescribed the plan.

18. The method of claim 17 further comprising:
further ranking the retrieved aural rehabilitation plans based on the actual success score for the retrieved plan corresponding to the first dimension.

19. The method of claim 11, wherein the rehabilitation plans in the database are indexed respectively by actual success scores, and wherein the selecting includes ranking the identified rehabilitation plans in view of the difference between the actual success score and the diagnostic

20. A system for selecting an aural rehabilitation plan for an individual having hearing loss, the system including a controller having communications capabilities and an aural rehabilitation plan database,
wherein the database includes, for respective physical and personal environment dimensions associated with hearing loss, profile information and scores corresponding to respective aural rehabilitation plans, and
wherein the controller, in response, to a search request for identification of a rehabilitation plan, the request including individual profile information and an individual score corresponding to at least a first of the physical and personal environment dimensions of an individual with hearing loss, searches the database to identify a plurality of rehabilitation plans corresponding to the at least first dimension and having profile information and a score matching the individual profile information and the individual score corresponding to the at least first dimension, wherein the individual profile information and the individual score are representative of measurement information obtained from an assessment of the least first dimension of the individual, wherein the scores are generated in view of diagnostic metrics defined from measurement information obtained from assessment of physical and personal environment dimensions and wherein the searching includes identifying aural rehabilitation plans for the first dimension having scores within a predetermined range of the individual score; and
wherein the controller supplies aural rehabilitation plan information corresponding to the identified aural rehabilitation plans.

21. The system of claim 20, wherein the controller, based on feedback generated from prescribing one of the identified plans to the individual, updates the database to include at least one of an annotation to the prescribed aural rehabilitation plan and a new aural rehabilitation plan indexed by the profile information and the score for the first dimension of the individual.

22. The system of claim 20, wherein the diagnostic metrics are defined by normalizing the measurement information obtained for each of the respective physical and personal environment dimensions into a linear scale signal metric and wherein the generated scores and the scores in the database each comprise at least one of a diagnostic score and a goal score.

23. The system of claim 20, wherein the personal environment dimensions include at least two of speech intelligibility, real world needs, critical success factors, localization, appearance and performance trade-offs and quality preferences.

24. The system of claim 20, wherein the database includes actual success scores and diagnostic scores indexing the respective aural rehabilitation plans corresponding to a set of the dimensions included in the database and wherein individual diagnostic scores are generated from measurement information obtained from assessment of the individual for the same set of dimensions included in the database, and
wherein the controller searches the database to retrieve aural rehabilitation plans having profile information and a cumulative diagnostic score matching the individual profile information and a cumulative individual diagnostic score, wherein the cumulative diagnostic score is a function of the diagnostic scores for the individual to whom a rehabilitation plan from the database identified as having matching profile information was prescribed, wherein the cumulative individual diagnostic score is a function of the individual diagnostic scores, wherein the retrieved aural rehabilitation plans are ranked based on cumulative actual success scores for the individuals to whom the retrieved rehabilitation plans were respectively prescribed, and wherein the cumulative actual success score for a retrieved plan is a function of the actual success scores for the individual prescribed the plan.

25. The system of claim 24 further comprising:
wherein the controller ranks the retrieved aural rehabilitation plans based on the actual success score for the retrieved aural rehabilitation plan corresponding to the first dimension.

26. The system of claim 20, wherein the aural rehabilitation plans in the database are indexed respectively by actual success scores, and wherein the controller ranks the identified aural rehabilitation plans in view of the difference between the actual success score and the diagnostic score corresponding to the identified aural rehabilitation plan.
